(19) United States
(12) Patent Application Publication
(10) Pub. No.: US 2001/0049528 A1
(43) Pub. Date: Dec. 6, 2001

(54) Implant screw

(76) Inventor: Kohei Kubota, Tokyo (JP)

Correspondence Address: ROSENMAN & COLIN LLP, 575 MADISON AVENUE, NEW YORK, NY, 10022-2585, US

(21) Appl. No.: 09/852,133

(22) Filed: May 9, 2001

(30) Foreign Application Priority Data
May 9, 2000 (JP) 2000-136109
May 12, 1999 (JP) H11-131954

(51) Int. Cl. A61B 17/76

(52) U.S. Cl. 606/65

(57) ABSTRACT

An implant screw assembly comprising; an inner screw provided with a thread on an outer periphery of one end, and with a base at the other end, wherein an angled socket for a wrench or a slot for a driver is provided at the base, an outer screw provided with a thread on at least part of an outer periphery, and coaxially disposed around the periphery of the base of the inner screw, wherein the thread of the inner screw and the thread of the outer screw are in the same rotation direction, and a meshing structure formed by an outer peripheral face of the inner screw and an inner peripheral face of the outer screw, to prevent relative rotation between the inner screw and the outer screw, whereby the outer screw and the inner screw are only able to slide axially.

### TECHNICAL FIELD OF THE INVENTION

The present invention relates to an interfragmental device used in osteosynthesis, and in particular relates to an implant screw which is inserted into cancellous bone of a skeleton to apply a compressive force to the fracture in it.

### BACKGROUND OF THE INVENTION

In osteosynthesis of the fracture, an implant screw is inserted so as to span between one bone portion (on a large distal bone fragment) such as a subtrochanteric portion or intertrochanteric portion and another bone portion (proximal bone fragment) such as a caput or femoral head, or a femoral neck including a caput or femoral head, and is fixed with appropriate repositioning to hasten synostosis in the fracture.

Heretofore there has been proposed an implant screw assembly comprising an inner screw with a thread at a tip end, and an outer screw coaxially arranged with the inner screw and having a through hole, into which the inner screw can be inserted, and a thread on an outer periphery. To enable rotation of each, the inner screw is provided with a groove for a driver or an angled socket for a wrench, while a head for a box wrench is provided on the outer screw.

The method of installation of this implant screw assembly in the fracture portion is as follows.

A bore for insertion of the outer screw is formed halfway into the canccllous bone from the bone cortex of one bone portion with respect to the fracture portion. Then a bore for insertion of the inner screw is further formed from the bottom of this bore so as to penetrate into the other bone portion through the fracture portion. Tapping to match the thread of the outer screw is performed in the opening portion of the bore, and tapping to match the thread of the inner screw is performed at the end portion of the bore.

Next, the inner screw is inserted into the bore, and rotated with a driver or the like, so that it is inserted to close to the cortex of the bone at the apical side. Then, the outer screw is inserted into the bore around and coaxial with the inner screw, and rotated with a box wrench or the like, and fastened with the head thereof protruding from the bore. That is to say, the inner screw and the outer screw are arranged coaxially, however these are not fixed to each other but are merely fixed to prevent displacement of the fracture portion.

From one to three of these implant screw assemblies are inserted and secured, so that there is no displacement of the fracture portion. Then, after a several days or months to several years follow-up, once synostosis is observed, the situation is judged and if necessary, all of the implant screw assemblies are extracted.

When this implant screw assembly is used, then by means of loading, or action and movement, a load is applied so as to compress on the fracture portion and hasten the synostosis. Since it is known that bone healing is hastened by applying compression to the fracture, then the aforementioned loading is used as the compression. However, with this implant screw assembly, there are problems such as, it takes time to sequentially insert the two screws, namely the inner screw and the outer screw, the fixing strength is weak because relative rotation can occur between the inner and outer screws, and a compressive force is not applied to the fracture at the time of screw insertion.

Moreover, to compress the fracture more strongly, a Herbert screw which had two threads of a different pitch at opposite ends has been proposed.

With the Herbert screw, the speed of insertion of the thread on the rear end side into the cancellous bone on the rear end side of the skeleton is slower than the speed of insertion of the thread on the front end side into the cancellous bone on the front end side of the skeleton. Therefore, the compressive force applied to the fracture, can be applied with the practitioner making adjustments. Furthermore, the time for inserting the two screws can be saved, and the extraction operation can be performed easily.

When the Herbert screw is used, this has the effect that the compressive force applied to the fracture can be fixed with one insertion. However, there are problems in that the force from outside is applied between the Herbert screw and the structure of the bone, which causes displacement of the screw or damage of the cancellous bone, and that the compressive force applied to the fracture is only for immediately after screw insertion (compression is not applied to the fracture after the screw has been inserted).

### SUMMARY OF THE INVENTION

To solve the above problems, it is an object of the present invention to provide an implant screw assembly which can be inserted without taking time and with excellent fixing strength, which can apply a compressive force to the fracture at the time of screw insertion and also after screw insertion, and which can be easily removed.

It is another object of the present invention to provide an implant screw assembly which makes it possible to release the force which comes from the outside.

### BRIEF DESCRIPTION OF THE DRAWINGS

FIG. 1 is a partial cut away, side elevational view showing a first embodiment of the implant screw assembly according to the present invention.

FIG. 2 is an exploded view of the implant screw assembly shown in FIG. 1 before assembling.

FIG. 3 is a longitudinal cross sectional view of the implant screw assembly in FIG. 2.

FIG. 4 is a cross-sectional view taken along the line IV-IV of FIG. 2.

FIG. 5 is a cross-sectional view taken along the line V-V of FIG. 2 wherein only the outer screw is illustrated.

FIG. 6 is a cross-sectional view taken along the line VI-VI of FIG. 2 wherein only the base is illustrated.

FIG. 7 is a cross-section view of two implant screw assemblies of the present invention inserted into a femoral neck portion.

FIG. 8 is a partially cut away, side elevational view of an implant screw assembly of another embodiment of the present invention.

FIG. 9 is an exploded, longitudinal cross sectional view of the implant screw assembly of FIG. 8 before assembling.

FIG. 10 is a partially cut away, side elevational view of a different embodiment of the present invention.

FIG. 11 is an exploded, longitudinal cross sectional view of the implant screw assembly of FIG. 10 before assembling.

FIG. 12 is a cross-section view taken along the line XII-XII of FIG. 10.

FIG. 13 shows a partially cut away, side elevational view of a different embodiment of the present invention.

FIG. 14 shows a partially cut away, side elevational view of yet another embodiment of the present invention.

FIG. 15 is a cross sectional view of an example of the double thread type.

FIG. 16 is a cross sectional view of an example of the combination of a single thread portion, double thread portion and a triple thread portion continued to each other.

FIG. 17 is a cross sectional view of a simple cylindrical outer screw fitted on an inner screw with a flange at one end.

FIG. 18 is a side elevational view of a base with an L-shaped slot to receive a protrusion formed on an inner screw.

### DETAILED DESCRIPTION OF THE EMBODIMENTS

The implant screw assembly in one embodiment of the present invention comprises an inner screw and an outer screw; the inner screw is provided with a thread on an outer periphery of one end, and with a base at the other end, wherein the inner screw preferably comprises a main body and a base integrally connected to the main body at the outer end of the main body, so that the base is separated from and fixed to the other end, and wherein an angled socket for a wrench or a slot for a driver is furnished at the base, and the outer screw is provided with a thread on at least part of an outer periphery, and coaxially disposed around the periphery of the base of the inner screw, wherein the thread of the inner screw and the thread of the outer screw are in the same rotation direction, and the implant screw further has a meshing structure for preventing relative rotation between the inner screw and the outer screw, and the meshing structure comprises meshing portions which are respectively provided on the outer peripheral face of the inner screw and the inner peripheral face of the outer screw so that the outer screw and the inner screw are only able to slide axially.

Alternatively, this screw assembly may comprise an inner screw provided with a thread on an outer periphery of one end, and with a base at the other end, the base being separated from and fixed to the other end as mentioned above, and an outer screw provided with a thread on at least part of an outer periphery, and furnished with an angled socket for a wrench or a slot for a driver, and coaxially disposed around the periphery of the base of the inner screw.

Moreover, the meshing portions of a non circular cross-section may be formed in a central portion, base or base portion in the inner screw, and in portions in the outer screw formed in a cross-section corresponding to this central portion, base or base portion.

The pitch of the thread of the outer screw may be shorter than the pitch of the thread of the inner screw, or the pitch of the thread of the outer screw may be the same as the pitch of the thread of the inner screw, and implant screw assemblies with different pitches are desirably used for different situations.

For force transmission, an engaging portion is provided on the inner peripheral portion of the outer screw to be engaged with the base of the inner screw so that a force can be transmitted between the inner screw and the outer screw. Thus a first engaging structure is formed for power transmission.

Furthermore, an engaging portion for preventing the removal of the outer screw from around the inner screw, may be provided on the inner peripheral portion of the outer screw, e.g. as in a stepped portion such that it is engaged with a protrusion or stepped portion provided on the base side of the thread of the inner screw, or a portion of the inner screw on the base side. Thus a second engaging structure is formed for preventing separation. Alternatively, the inner periphery of the outer screw may be made greater than the outer periphery of the thread of the inner screw. Thus, either a condition with the outer screw attached around the periphery of the inner screw, or a condition with the outer screw detached from the inner screw is possible.

A bore in the inner screw may be provided through the axial center at least at a base portion to which the base is connected. And, a thread may be provided on the inner periphery of the bore through the axial center in the base portion of the inner screw. The mating member of this thread in the bore is a screw assembly as explained next.

This screw assembly is applied to the assembly of inner and outer screws, and may have a compressive force adjusting screw with a screw portion for engaging with the thread provided in the bore through the axial center in the inner screw, and a flange portion for abutting against the outer screw around the inner screw by advancing the screw portion into the thread in the inner screw, so that the relative position of the inner screw with reference to the outer screw is slidable by advancing the screw portion of the compressive force adjusting screw into the thread of the inner screw as mentioned above.

The thread of the inner screw and the thread of the outer screw may be of the single thread type, or of the double or dual thread type, or of the triple thread type. In addition, the thread may comprise a single thread portion and double thread portion and sometimes a triple thread portion continued to each other.

The thread of the inner screw and the thread of the outer screw may be furnished with a self tapping device and a reverse self tapping device.

The thread of the inner screw or the thread of the outer screw may be in a taper shape.

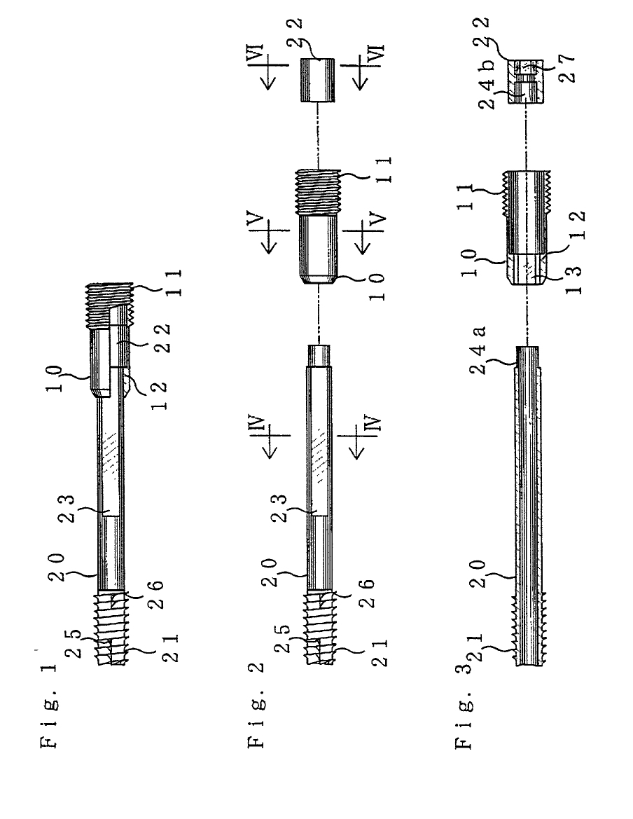

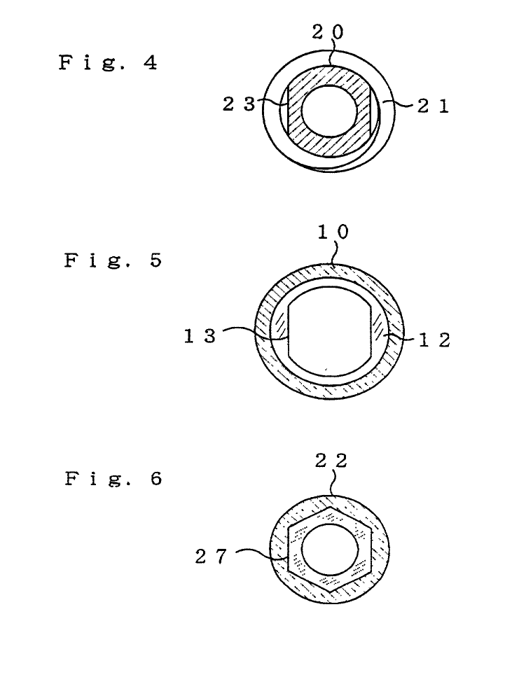

Now, the present invention will be described based on the embodiments in the drawings. FIG. 1 is a partial cut away side view showing a first embodiment. FIG. 2 is an assembly drawings of the embodiment shown in FIG. 1. FIG. 3 is a longitudinal cross sectional view of FIG. 2. FIG. 4 is a cross-sectional view taken along the line IV-IV of FIG. 2. FIG. 5 is a cross-sectional view taken along the line V-V of FIG. 2 wherein only the outer screw is illustrated. FIG. 6 is a cross-sectional view taken along the line VI-VI of FIG. 2 wherein only the base is illustrated.

The implant screw assembly of the present embodiment comprises a cylindrical inner screw 20 and a tube-shaped outer screw 10. The inner screw 20 comprises a main body provided with a thread 21 on an outer periphery of one end, and a base 22 connected to the main body at the other end, wherein the base 22 is separated from and fixed to the other end as detailed later. And, an angled socket 27 for a wrench or a slot for a driver (not shown in the figure) is furnished at the base 22. The tube-shaped outer screw 10 is provided with a thread 11 coaxially disposed around the periphery of the base 22. The thread 21 of the inner screw 20 and the thread 11 of the outer screw 10 are formed in the same rotation direction.

A meshing structure for stopping relative rotation between the inner screw 20 and the outer screw 10 comprises meshing portions 23, 13 which are respectively provided on the outer peripheral face of the inner screw 20 and the inner peripheral face of the outer screw 10. Consequently, the outer screw 10 and the inner screw 20 rotate together as one body, and are only able to slide axially. With the embodiment shown in the figure, the meshing portions 23 arc flats formed on the opposite sides of the cylindrical portions (refer to FIG. 4) while the meshing portions 13 are flat protrusions facing the meshing portions 23 (refer to FIG. 5).

The material of the outer and inner screws 10, 20 is desirably a titanium alloy having high strength, affinity, and corrosion-resistance.

The main body of the inner screw 20 is formed in a circular tube shape for example of 6 mm OD×3.4 mm ID×90 mm long, and provided with the thread 21 of a maximum diameter 8 mm, and 1.75 mm pitch, and with the meshing portions 23 formed parallel to each other and spaced 5 mm apart from each other (a corresponding meshing portion is provided on the other side in FIG. 1 and 2), and the base 22 has a size of 7.2 mm diameter×11 mm and formed with an M5 hexagonal socket 27 in one end.

A bore extending through the axial center of the inner screw 20 is used for passing a 3 mm diameter guide pin. With an implant screw assembly which is small in diameter and short in length, a guide pin is not required for insertion in the bone, and then the inner screw for such an implant screw assembly may be solid without the bore.

The outer screw 10 is a circular tube of for example 9 mm OD×7.25 mm ID×30 mm long, provided with the thread 11 of 11 mm maximum OD with a 1.5 mm pitch, being smaller than the pitch of the thread 21 of the inner screw 20. The engaging portion 12 is formed in 6.05 mm diameter so that the inner screw 20 can be inserted thereinside, and provided with parallel flat meshing portions 13 which are 5.05 mm apart. The meshing portions 13 are positioned on opposite sides of the meshing portions 23 of the inner screw 20, to form a meshing structure thereby preventing relative rotation between the inner screw 20 and the outer screw 10. The implant screw assembly has a maximum length of approximately 95 mm.

As will be understood from the abovementioned dimensions, the inner circumference of the engaging portion 12 of the inner peripheral face of the outer screw 10 is smaller than the outer circumference of the base 22 of the inner screw 20. Specifically, the outer circumference of the base 22 is formed in a stepped shape with reference to the main portion of the inner screw 20, and the inner circumference of the engaging portion 12 is formed in a stepped shape to abut the outer circumference of the base 22. Consequently, by abutting the engaging structure 12 of the outer screw 10 with the base 22, the inner screw 20 cannot be removed in the leftward direction of FIG. 1. Furthermore, by abutting the meshing portions 13 of the outer screw 10 with the end portions of the meshing portions 23 of the inner screw 20, the inner screw 20 cannot be removed in the rightward direction of FIG. 1. Specifically, the end of meshing portion 23 is formed in a stepped shape between the flat surface of the meshing portion 23 and the cylindrical surface of the inner screw 20, and the meshing portion 13 of the outer screw 10 is abutted to the stepped portion. These are the second engaging structure. The outer screw 10 is thus slideable axially over a fixed range with respect to the inner screw 20.

Alternatively, the construction may be such that the meshing portions 23 of the inner screw 20 extend as far as the thread 21, and the sliding of the outer screw 10 is limited and its removal prevented, by abutting the meshing portions 13 of the outer screw 10 against the end portion of the thread 21. Any protrusion can be formed on the main body of the inner screw 20 to which the meshing portions 13 can be abutted.

On the other hand, the outer screw 10 can be made in a single cylindrical shape and abutted to the flange portion 29 of the base of the inner screw 20 as in FIG. 17.

In addition, any protrusion (not shown) can be formed on the inner screw near the base, and the outer screw 10 can be formed with a L-shaped slot 19 to receive the protrusion as in Fig. 18.

Alternatively, the thread 21 of the inner screw 20 may be made smaller than the internal circumference of the outer screw 10 (including the engaging structure 12). In this case, the inner screw 20 can be removed in the rightward direction in FIG. 1 and disconnected from the outer screw 10. By having such a disconnection capability, the assembly operation as later mentioned is no longer required, enabling a low cost construction, and ease of handling.

The thread 21 of the inner screw 20 may be provided with a self tapping mechanism 25 and a reverse self tapping mechanism 26. While not shown in the figure, the thread 11 of the outer screw 10 may also be provided with a similar self tapping mechanism and a reverse self tapping mechanism.

The assembly method of the implant screw of the present invention comprises the steps of fitting the outer screw 10 over the inner screw 20 with the meshing portions 13 of the outer screw 10 facing towards the meshing portions 23 of the inner screw 20, and then securing the base 22 by a known technique such as fitting and crimping a crimping portion 24b of the base 22 over a crimping portion 24a of the inner screw 20 or bonding the base 22 to the inner screw 20.

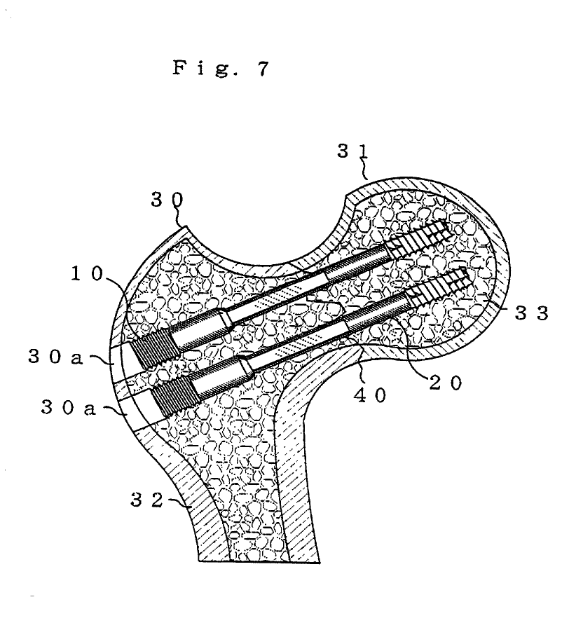

A method of inserting the implant screw assembly of the present invention into the femoral neck portion is described hereunder. FIG. 7 is a cross-section view of two implant screw assemblies of the present invention inserted into the femoral neck portion.

A fracture portion 40 is reduced anatomically, and bores 30a to match the outer screw 10 are drilled in the cancellous bone 33 at two locations from the outside cortex 32 of the femoral neck portion 30, and the bores 30a are continued to bores to match the inner screw 20 drilled in the cancellous bone 33. Alternatively, at first a guide pin (not shown in the figure) may be inserted, so that a bore for the outer and inner screws is drilled in conformance with the guide pin. The guide pin is then removed after insertion of the implant screw assembly of the present invention.

Next, a thread is tapped along the bore 30a near the opening thereof to match the thread of the outer screw 10, and a thread is tapped along the bottom end portion of the bore 30a to match the inner screw 20. Then, a wrench (not shown in the figure) is inserted in the angled socket 27 (FIG. 3), and the inner screw 20 is screwed into the bore 30a. Since the construction is such that the outer screw 10 does not rotate relative to the inner screw 20, then by screwing in the inner screw 20, the outer screw 10 is also screwed in. From the position where the base 22 of the inner screw 20 abuts against the engaging portion 12 of the outer screw 10 (refer to FIG. 1), a compressive force can be applied to the fracture portion 40. Hence a compressive force is adjusted with the force of rotating the wrench to thus complete the insertion. The outer screw 10 may be in a condition protruding from the cortex 32, or held with its tip end stopped in the cortex, or embedded as far as the cancellous bone.

This has the effect that the compressive force on the fracture portion 40 continues to he maintained, and the inner screw 20 and the outer screw 10 are abutted only at the engaging portion 12 of the outer screw 10 and the base 22 of the inner screw 20. Therefore, when a force is applied from outside, the engaging portion 12 and the base 22 are separated from each other, so that the force from the outside does not act between the implant screw assembly and the structure of the bone.

If the implant screw assembly with the self tapping device 25 as shown in FIG. 1 and 2 is used, then the beforementioned tapping operation is not necessarily required, and the bore can be made easily.

Furthermore, after follow-up, once the synostosis of the fracture portion 40 has been observed, the implant screw assembly of the present invention can be extracted. At this time, if the reverse self tapping device 26 is provided, then the implant screw assembly can easily withdrawn.

Moreover, in another embodiment of the present invention, the pitch of the thread 11 of the outer screw 10 and the pitch of the thread 21 of the inner screw 20 are made the same to each other (not shown in the figure). When such an implant screw assembly where the pitch of the two threads 11 and 20 are the same is inserted as described before, the compressive force is not applied to the fracture portion. Consequently, by simultaneously using such an implant screw assembly together with the aforementioned implant screw assembly where the pitch of the thread 11 is less than the pitch of the thread 21, an affect can be obtained that the compressive force applied to the fracture portion can be adjusted by one implant screw.

Furthermore, in the case where displacement of the fracture is minimal, there is also the case where it is preferable to apply practically no compressive force to the fracture portion. In this case also, an implant screw assembly of this embodiment where the pitch of the two threads 11 and 21 are the same is used. Since the inner screw 20 and the outer screw 10 have a relation where only the engaging portion 12 and the base 22 are engaged with each other, then in the condition that the compressive force is not applied to the fracture portion, during follow-up it is possible to perform a medical treatment where by some other method, a large compressive force is applied to the fracture portion.

It is desirable that the compressive force applied to the fracture portion is optionally designed by the pitch of the two threads.

The bone is not uniform in property and, its structure and strength change even in a millimeter range, or for example there is a transition from the cancellous bone to the bone cortex. Accordingly one thread must sometimes span between e.g. the cancellous bone to the bone cortex. Therefore, it is desired that implant screws have a changing thread pattern to correspond to the changing property of the bone, in two or three threads with different sizes and shapes, or even in two or three different thread portions in a continuous thread, which provides excellent fixing performance, pitch adjustment, thread length control and press force control,

For example, an implant screw can have a single thread portion at the front end and a dual thread portion at the rear end, and set in the bone with the fracture located between the single thread portion and the dual thread portion and with the single thread portion on the front side located on the coarse structure portion of the bone and with the dual thread portion (smaller pitch portion) on the rear side located on the dense structure portion. This prevents the fracture from being subjected to strong press force when setting the implant screw in the bone. On the other hand, another implant screw can have a dual thread portion at the front end and a single thread portion at the rear end, and set in the bone with the fracture located between the dual thread portion and the single thread portion and with the dual thread portion (smaller pitch portion) on the front side located on the dense structure portion of the bone and with the single thread portion on the rear side located on the coarse structure portion. This can apply increased press force to the fracture when setting the implant screw in the bone. When the same single thread portion type is used on the both rear and front ends of the implant screw, for example if the rear end is made longer for fixing, the fracture will be subjected to a large press force.

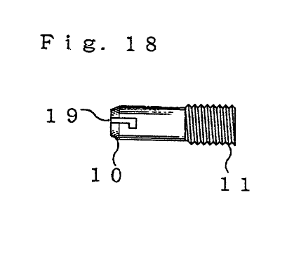

Accordingly, the thread 21 can be formed in a single thread type as shown in the previous figures or in a double thread type as shown in FIG. 15 for the inner screw 20 or in a triple thread type. In addition, the thread 21 can be made such that a single thread type portion can be continued to a double thread type portion and even to a triple thread type section as shown in FIG. 16 for the inner screw 20. Furthermore, the length, number, and thickness of the implant screw assembly may be appropriately selected to suit the shape and condition of the affected part.

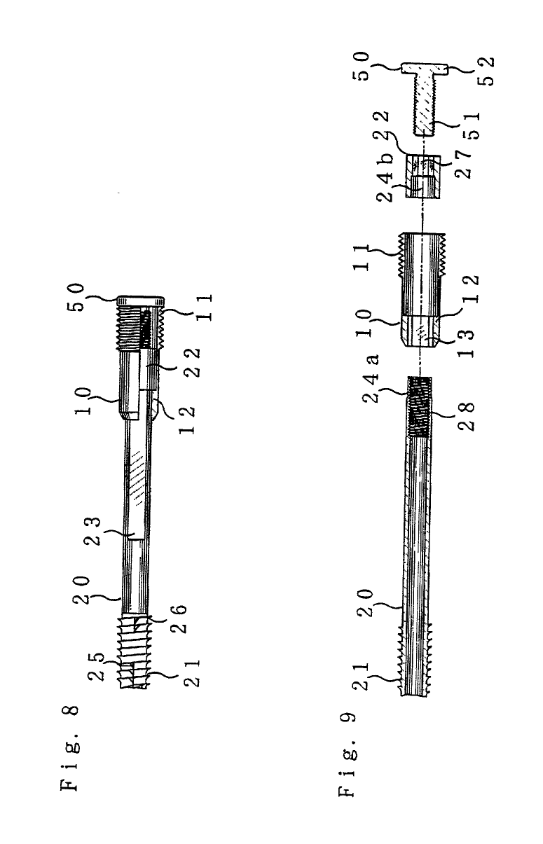

A partially cut away section side view of an implant screw assembly of another embodiment is shown in FIG. 8, while a longitudinal assembly view is shown in FIG. 9.

In the implant screw assembly of this embodiment, a compressive force adjusting screw 50 is further provided in the structure as disclosed in the aforementioned embodiment. Furthermore, a thread 28 is provided on the inner periphery of the inner screw 20. On the compressive force adjusting screw 50 there is provided a screw portion 51 for engaging with the thread 28, and a flange portion 52. The flange portion 52 may be formed in a hexagonal shape to take a box wrench, or the flange portion 52 may be provided with an angled socket for a wrench or with a groove for a driver. By tightening the screw portion 51 into the thread 28, the flange portion 52 of the compressive force adjusting screw 50 abuts against the outer screw 10, so that the base 22 of the inner screw 20 is separated from the engaging portion 12 of the outer screw 10, thereby enabling the compressive force applied to the fracture portion to be increased. When the aforementioned force from the outside is applied, the flange portion 52 is separated from the outer screw 10, so that the force from the outside would not act between the implant screw and the structure of the bone.

It is useful if the compressive force adjusting screw 50 is provided when the implant screw assembly is installed in an affected part, however this may be fitted during follow-up.

When the implant screw assembly of this invention is withdrawn, if a wrench with a bore formed on the center of rotation is used and an auxiliary tool is secured to the thread 28, this also has the effect that the rotation of the implant screw assembly is facilitated.

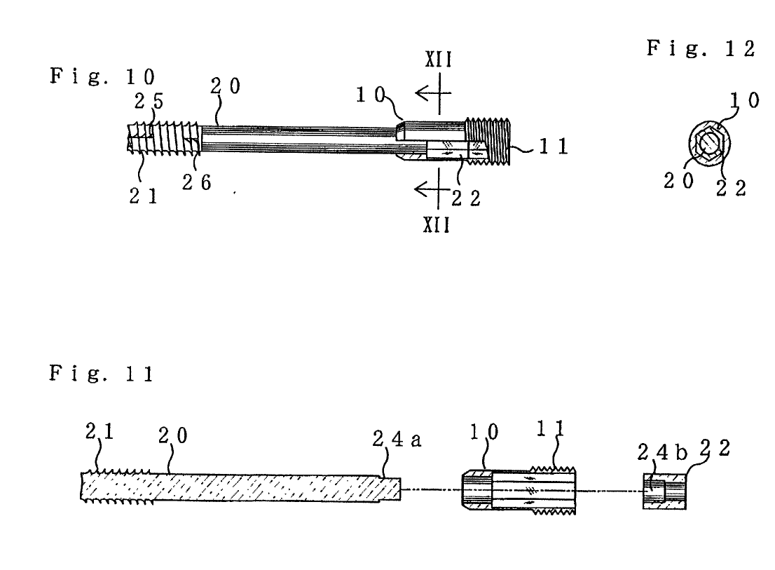

FIG. 10 is a partially cut away side view of a different embodiment of the present invention, while FIG. 11 shows a cross-section view of the assembly. FIG. 12 is a cross-section view taken along the line XII-XII of FIG. 10. As shown in the figures, the inner screw 20 is a simple cylinder shape and the cross-section of the outer peripheral face of the base 22 of the inner screw 20 is for example a hexagon shape. Similarly the cross-section of the inner peripheral face of the outer screw 10 is a hexagon shape. Instead of the meshing structure comprising the meshing portions 13 (FIG. 3) and the meshing portions 23 (FIG. 2), the meshing structure of this example comprises the outer peripheral face of the base 22 and the inner peripheral face of the outer screw 10 to prevent relative rotation between the inner screw 20 and the outer screw 10. Furthermore, the inner peripheral face of the outer screw 10 also serves as an angled socket for a wrench, and hence there is no longer the need for the angled socket 27 (FIG. 3) provided in the base 22 of the inner screw 20. The shapes of the other parts are the same as for the aforementioned embodiments.

When as with this embodiment, the meshing structure is provided on the base of the inner screw, and the hexagonal socket for rotation has a double purpose, manufacture is simplified. Furthermore, a groove for a driver may be provided on the end portion of the outer screw.

At the time of actual implanting, by applying force to the outer screw in this way to rotate it, the implant screw assembly can be easily inserted into the bone.

Furthermore, the securing force on the bone may be increased by tapering the outer screw in a convergent shape. Moreover, only the thread of the outer screw may be tapered in a convergent shape.

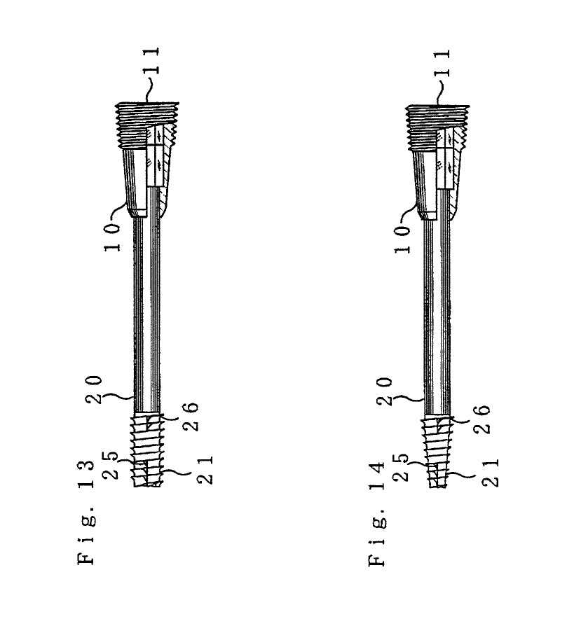

FIG. 13 shows a partially cut away side view of a different embodiment of the present invention. With this embodiment, the outer screw assembly is formed with a taper. Consequently, the thread of the outer screw is also tapered.

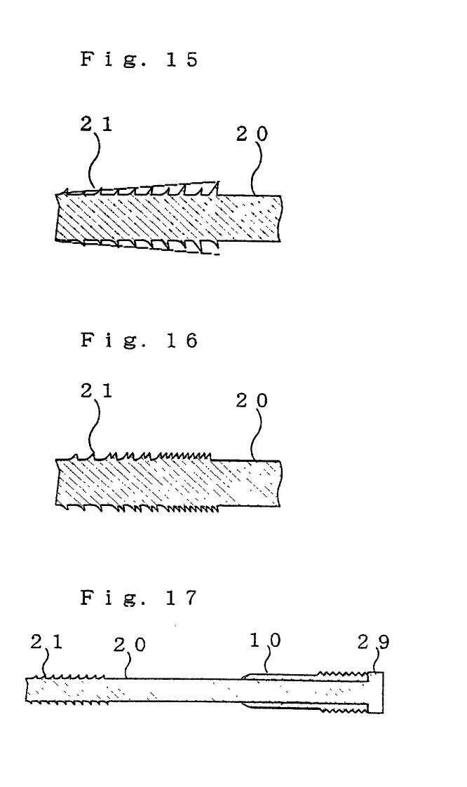

Furthermore, FIG. 14 shows a partially cut away side view of yet another embodiment. With this embodiment, the thread portion of both the outer screw and the inner screw are tapered.

In this way, since the thread is tapered, the thread cuts into the bone, so that the securing force on the bone can be increased.

The present invention is not limited to the parts, materials and shape of the illustrated embodiments, which are merely used for explaining the present invention.

As described in detail above, the implant screw assembly of the present invention can be inserted by one operation. Furthermore, at the time of insertion and after insertion, an optional compressive force can be applied to the fracture portion. Moreover, it is possible to avoid the situation where a force coming from the outside has a harmful effect on the condition between the implant screw assembly and the structure of the bone.

If the outer screw and the inner screw are made separable or removable, then the outer screw can be set from the front end, rear end or middle of the inner screw, and manufacturing cost can be reduced, while if the outer screw is assembled integral with the inner screw, the insertion into the affected part is facilitated. In order to be separable or removable, the outer screw can be made in halves.

Moreover, by providing a self tapping device, the tapping operation prior to insertion can be omitted, and by providing a reverse self tapping device, extraction is facilitated.

By simultaneously using an implant screw assembly with the pitch of the two threads the same together, with an implant screw assembly where the pitch of the thread of the outer screw is less than the pitch of the thread of the inner screw, then the compressive force applied to the fracture portion can be adjusted with one implant screw assembly, so that there is no excessive compressive force.

Furthermore, when the meshing structure of the inner screw and the outer screw is provided in the inner peripheral face of the outer screw and the base of the inner screw, if the cross-section shape is hexagonal, then the bore can also serve the purpose as an insertion socket for a hexagonal wrench. Moreover, insertion in the bone is facilitated.

In addition, if the thread is formed with a taper, this has the affect that the securing force to the bone is increased.

On the other hand, the inner screw and outer screw can be rotated relative to each other such that a driver or wrench is inserted into the axial end of the inner screw to rotate it with the outer screw kept stationary.

What is claimed is:

1. An implant screw assembly comprising; an inner screw provided with a thread on an outer periphery of one end, and with a base at the other end, wherein an angled socket for a wrench or a slot for a driver is provided at the base, an outer screw provided with a thread on at least part of an outer periphery, and coaxially disposed around the periphery of the base of the inner screw, wherein the thread of the inner screw and the thread of the outer screw are in the same rotation direction, and a meshing structure formed by an outer peripheral face of the inner screw and an inner peripheral face of the outer screw, to prevent relative rotation between the inner screw and the outer screw, whereby the outer screw and the inner screw are only able to slide axially.

2. The implant screw assembly of claim 1, wherein the pitch of the thread of the outer screw is shorter than the pitch of the thread of the inner screw.

3. The implant screw assembly of claim 1, wherein the pitch of the thread of the outer screw is the same as the pitch of the thread of the inner screw.

4. The implant screw assembly of one of claims 1 to 3, wherein a first engaging structure is formed by the base of the inner screw and an inner peripheral portion of the outer screw engaged with the base, so that a force can be applied between the inner screw and the outer screw.

5. The implant screw assembly of one of claims 1 to 3, wherein a second engaging structure for preventing the removal of the outer screw from around the inner screw, is formed by a protrusion or stepped portion provided on the opposite sides of the inner screw, and an inner peripheral portion of the outer screw engaged with the protrusion or stepped portion.

6. The implant screw assembly of one of claims 1 to 4, wherein the inner periphery of the outer screw is made greater than the outer periphery of the thread of the inner screw, so that the outer screw is detached from the inner screw is possible.

7. The implant screw assembly of one of claims 1 to 6, wherein a bore through the axial center in the inner screw is provided at least at a base portion associated with the base.

8. The implant screw assembly of claim 7, wherein a thread is provided on the inner periphery of the bore through the axial center in the base portion of the inner screw, so that the thread receives a screw inserted from outside.

9. The implant screw assembly of claim 8, wherein a compressive force adjusting screw is provided with a screw portion for engaging with a thread provided in the bore in the inner screw, and a flange portion for abutting against the outer screw by advancing the screw portion into the thread, so that the relative position of the inner screw with reference to the outer screw is slidable by advancing the screw portion into the thread.

10. The implant screw assembly of one of claims 1 to 9, wherein the thread of the inner screw and the thread of the outer screw is provided with a self tapping device and a reverse self tapping device.

11. The implant screw assembly of claim 1, wherein the thread of the inner screw or the thread of the outer screw is formed in a taper shape.

12. An implant screw assembly comprising an inner screw provided with a thread on an outer periphery of one end, and with a base at the other end, and an outer screw provided with a thread on at least part of an outer periphery, and furnished with an angled socket for a wrench or a slot for a driver, and coaxially disposed around the periphery of the base of the inner screw, wherein the thread of the inner screw and the thread of the outer screw are rotated in the same direction and a meshing structure is formed by an outer peripheral surface of the inner screw and an inner peripheral surface of the outer screw to prevent relative rotation of the inner and outer screws, whereby the outer and inner screws are only able to side axially.

13. The implant, screw assembly of claim 12, wherein the meshing structure is formed by a central portion, base, or base portion of a non circular cross-section in the inner screw, and a portion in the outer screw having a cross-section corresponding to the non circular cross-section in the inner screw.

14. The implant screw assembly of one of claim 1 and 12, wherein at least one of the inner screw and outer screw is of the single thread type while the other is of the dual thread type.

15. The implant screw assembly of one of claim 1 and 12, wherein at least one thread formed in the inner screw and outer screw has at least two thread portions selected from the group of single thread type, double thread type and triple thread type.
